(19) United States
(12) Patent Application Publication
(10) Pub. No.: US 2022/0152151 A1
(43) Pub. Date: May 19, 2022

(54) ACELLULAR INTRAVENOUS INFUSION INCLUDING MESENCHYMAL STEM CELL GROWTH FACTORS AND EXOSOMES

(71) Applicant: DIRECT BIOLOGICS, LLC, Austin, TX (US)

(72) Inventor: Kenneth Allen PETTINE, Fort Collins, CO (US)

(21) Appl. No.: 17/432,138

(22) PCT Filed: Feb. 19, 2020
(86) PCT No.: PCT/US2020/018821

§ 371 Date: Aug. 19, 2021

Related U.S. Application Data
(60) Provisional application No. 62/807,610, filed on Feb. 19, 2019.

(51) Int. Cl. A61K 38/18; A61K 9/00

(52) U.S. Cl. CPC A61K 38/18; A61K 9/0019

(57) ABSTRACT

A therapeutic intravenous (IV) infusion for the treatment of various medical conditions may include acellular mesenchymal stem cell (MSC) derived growth factors and exosomes. The acellular MSC derived growth factors and exosomes may include cells or cell conditioned media cultured under normal hyperoxic culturing conditions and cells cultured under harsh wound healing conditions.

This application claims the benefit of U.S. Provisional Application No. 62/807,610, filed on Feb. 19, 2019, which is incorporated herein by reference in their entirety.

### I. BACKGROUND

MSCs, after their initial discovery in bone marrow, have been isolated and characterized from several adult and fetal tissues, including adipose (fat), dermis (skin), synovial fluid, periosteum, umbilical cord blood, placenta, and amniotic fluid. MSCs are partially defined by their ability to differentiate into tissues including osteoblasts (bone cells), chondrocytes (cartilage cells), myocytes (muscle cells), and adipocytes (fat cells).

While allogenic cellular MSC IV treatments have been widely pursued, there are numerous safety and regulatory concerns surrounding allogenic cellular preparations. The inherent problems with IV infusions of living MSCs include the trapping of the cells in the lungs, causing the cells to die within 24 hours. The cellular debris from this cell death ends up in the liver to be disposed. Current autogenous treatments from bone marrow concentrate only deliver a few thousand MSCs. While allogenic expanded MSC IV infusions can deliver hundreds of millions of living MSCs, they all get trapped in the lungs and die. The long term effects of introducing the foreign DNA into the recipient is unclear and questions have arisen on whether introducing the large amount of foreign DNA could be carcinogenic. Therefore, what is needed is an acellular therapeutic IV infusion product for treatment various autoimmune conditions, decreasing systemic inflammation, and promoting rapid healing from acute traumatic soft tissue orthopedic injuries, while simultaneously avoiding the potential negative long term effects associated with introducing cellular living MSCs into a recipient.

### II. SUMMARY

The embodiments described herein relate generally to medical treatments, and more particularly, to an acellular intravenous (IV) infusion including mesenchymal stem cell (MSC) growth factors and exosomes for the treatment of various medical conditions. Accordingly, in one aspect, disclosed herein are therapeutic intravenous (IV) infusion or injection compositions for the treatment of various medical conditions, the therapeutic IV infusion or injection comprising: acellular mesenchymal stem cell (MSC) derived growth factors and/or exosomes.

Some embodiments of the present disclosure include a therapeutic intravenous (IV) infusion or injection for the treatment of various medical conditions. The IV infusion may include acellular mesenchymal stem cell (MSC) derived growth factors and exosomes. The acellular MSC derived growth factors and exosomes may include cells or cell conditioned media cultured under normal hyperoxic culturing conditions and cells cultured under harsh wound healing conditions. The therapeutic volume of the IV infusion may be used for the treatment of various medical conditions, including fibromyalgia, multiple sclerosis, Parkinson's disease, Crohn's disease, acute orthopedic soft tissue injuries, liver pathology due to alcohol use, and the like.

In one aspect, disclosed herein are methods of any preceding aspect, wherein the therapeutic IV composition comprises MSC derived growth factors (such as, for example, prostaglandin E2 (PGE2), transforming growth factor β1 (TGF-β1), hepatocyte growth factor (HGF), stromal cell derived factor-1 (SDF-1), nitric oxide, indoleamine 2,3-dioxygenase, interleukin-4 (IL-4), IL-6, interleukin-10 (IL-10), IL-1 receptor antagonist and soluble TNF-α receptor, insulin-like growth factors, fibroblast growth factors (FGF) 1-23 (especially, FGF1 and FGF2), bone morphogenetic proteins (BMPs) 1-15, epidermal growth factor (EGF), transforming growth factor-α (TGF-α) macrophage-stimulating protein (MSP), platelet derived growth factor (PLGF), vascular endothelial growth factor (VEGF), macrophage colony stimulating factor (M-CSF), insulin, granulocyte colony stimulating factor (G-CSF), granulocyte macrophage colony stimulating factor (GM-CSF), and/or hormones including estrogen, and thyroid hormones) obtained from MSC.

Also disclosed herein are methods of treating, reducing, inhibiting, ameliorating, and/or preventing a cancer or metastasis, aplastic anemia, Plasma cell disorders, Inborn errors of metabolism, immune deficiencies, POEMS syndrome, Fibromyalgia, Multiple sclerosis, Parkinson's disease, Crohn's disease, Spinal cord injury, Acute orthopedic soft tissue injuries, Neuropathy, Liver pathology due to alcohol use, Lyme disease, or primary amyloidosis in a subject comprising administering to the subject IV infusion composition of any preceding aspect.

In one aspect, disclosed herein are the methods of treating, reducing, inhibiting, ameliorating, and/or preventing a cancer and/or metastasis of any preceding aspect, wherein the cancer comprises acute leukemia, Adrenoleukodystrophy, Aplastic anemia, Bone marrow failure syndromes, Chronic leukemia, Hemoglobinopathies, Hodgkin's lymphoma, Multiple myeloma, Myelodysplastic syndromes, Neuroblastoma, Non-Hodgkin's lymphoma.

### III. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “subject” is defined herein to include animals such as mammals, including, but not limited to, primates (e.g., humans), cows, horses, pigs, sheep, goats, dogs, cats, rabbits, rats, mice and the like. In some embodiments, the subject is a human.

“Administration” to a subject includes any route of introducing or delivering to a subject an agent. Administration can be carried out by any suitable route, including oral, topical, intravenous injection, intravenous infusion, subcutaneous, transcutaneous, transdermal, intramuscular, intra joint, parenteral, intra-arteriole, intraarticular, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

A “decrease” can refer to any change that results in a smaller gene expression, protein production, amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease, disorder, injury, or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

The terms “prevent,” “preventing,” “prevention,” and grammatical variations thereof as used herein, refer to a method of partially or completely delaying or precluding the onset or recurrence of a disease and/or one or more of its attendant symptoms or barring a subject from acquiring or reacquiring a disease or reducing a subject's risk of acquiring or reacquiring a disease or one or more of its attendant symptoms.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain (i.e., nociception) relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Composition

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular therapeutic IV composition comprising MSC derived exosomes and/or growth factors is disclosed and discussed and a number of modifications that can be made to a number of molecules including the therapeutic IV composition are discussed, specifically contemplated is each and every combination and permutation of therapeutic IV composition comprising MSC derived exosomes and/or growth factors and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

The product of the present disclosure may be as a therapeutic intravenous (IV) infusion including mesenchymal stem cell (MSC) derived growth factors and exosomes for the treatment of various medical conditions and may comprise the following elements. This list of possible constituent elements is intended to be exemplary only, and it is not intended that this list be used to limit the product of the present application to just these elements. Persons having ordinary skill in the art relevant to the present disclosure may understand there to be equivalent elements that may be substituted within the present disclosure without changing the essential function or operation of the product.

By way of example, some embodiments of the invention include a therapeutic intravenous (IV) infusion for the treatment of various medical conditions, the therapeutic IV\ infusion comprising acellular mesenchymal stem cell (MSC) derived growth factors and exosomes.

The primary trophic property of MSCs is the secretion of growth factors and exosomes to induce cell proliferation and angiogenesis. Exosomes express mitogenic proteins such as transforming growth factor-alpha (TGF-α), TGF-β, hepatocyte growth factor (HGF), epithelial growth factor (EGF), basic fibroblast growth factor (FGF-2) and insulin-like growth factor-1 (IGF-1) to increase fibroblast, epithelial and endothelial cell division. Vascular endothelial growth factor (VEGF), IGF-1, EGF and angiopoietin-1 are released to recruit endothelial lineage cells and initiate vascularization. It has been hypothesized that an individual's genotype has a role in the expression of and reaction to these Growth Factors, providing credence to the philosophy of personalized medicine utilizing genetically matched donors and recipients. Regarding the anti-inflammatory and immunomodulatory properties of MSCs, it is believed that, in many types of musculoskeletal trauma, inflammatory conditions at the site of injury impede the natural repair processes by local progenitor and mature cells. Without being bound by theory, it is believed that MSCs assist via paracrine mechanisms and modulate the regenerative environment via anti-inflammatory and immunomodulatory mechanisms. In response to inflammatory molecules such as interleukin-1 (IL-1), IL-2, IL-12, tumor necrosis factor-α (TNF-α) and interferon-gamma (INF-γ), MSCs secrete an array of growth factors and anti-inflammatory proteins with complex feedback mechanisms among the many types of immune cells. The key immunomodulatory cytokines include prostaglandin 2, TGF-β1, HGF, SDF-1, nitrous oxide, indoleamine 2, 3-dioxygenase, IL-4, IL-6, IL-10, IL-1 receptor antagonist and soluble tumor necrosis factor-a receptor. MSCs prevent proliferation and function of many inflammatory immune cells, including T-cells, natural killer cells, B-cells, monocytes, macrophages, and dendritic cells. Although MSCs across species are able to regulate T-cell activity, the mechanisms are not identical across mammalian species.

A characteristic of chronically inflamed environments is a persistent imbalance in the types of helper T-cells and macrophages. MSC exosomes indirectly promote the transition of TH1 to TH2 cells by reducing INF-γ and increasing IL-4 and IL-10. The restored TH1/TH2 balance has been shown to improve tissue regeneration in cartilage, muscle, and other soft tissue injuries, alleviate symptoms of autoimmune diseases, and have an anti-diabetic effect. Similarly, reduction in INF-γ and secretion of IL-4 promotes a shift in macrophages from M1 (proinflammatory, anti-angiogenic and tissue growth inhibition) to M2 (anti-inflammatory, proremodeling and tissue healing) type, an effect required for skeletal, muscular, and neural healing and regeneration.

Studies have shown that allogenic cellular MSC IV treatments can be beneficial for the treatment of numerous medical conditions, such as multiple sclerosis (MS), amyotrophic lateral sclerosis (ALS), Parkinson's disease, fibromyalgia, rheumatoid arthritis, Crohn's disease, and the like. Such treatments can also be beneficial in treating elevated liver enzymes from alcohol consumption. It has also been shown that MSC treatments have the ability to regenerate damages tissue. Combined with their capacity to regulate immune and inflammatory responses, this provides for MSC treatments likely being a beneficial treatment option for autoimmune diseases.

The MSC derived growth factors and exosomes used in the disclosed therapeutic IV compositions can include at least one member selected from the group consisting of cells or cell conditioned media cultured under normal hyperoxic culturing conditions and cells cultured under harsh wound healing conditions. Hyperoxic culturing conditions may be defined as about 21%, wherein about 21% may be 21%±5%, oxygen with serum supplements and oxygen, while wound healing conditions may be defined as about 1 to about 5% oxygen in the presence of inflammatory cytokines, angiogenic factors, and/or reduced glucose.

A particular method for making the intravenous product of the present disclosure includes preparing a growth factor powder additive comprising autogenous or allogenic MSC growth factors and exosomes; culturing the MSCs to create a media under normal hyperoxyic culturing conditions or under wound healing hypoxic conditions, including reduced oxygen and glucose; collecting and combining the conglomerate mixture with a sugar solution and freezing the conglomerate mixture, wherein the conglomerate mixture may comprise exosomes, peptides, proteins, cytokines, growth factors, extracellular matrix (ECM), and chemokines from human or animal MSCs, multipotential stromal cells, fibroblasts or other cells; and lyophilizing the conglomerate mixture to create a dry powder ingredient. The dry powder mixture may then be introduced into an IV fluid for the administration of the autogenic or allogenic MSC derived exosomes via IV infusion.

In some embodiments, it may be beneficial to create a stem cell growth factor and exosome composition for a specific pathology, unique complexion challenges, race, or ethnicity. In such instances, the method may comprise identifying a single nucleotide polymorphism (SNPs) of a potential donor to match that of an end user; obtaining MSCs from the donor having the same or similar SNP profile as the end user; and preparing an MSC derived growth factor and exosomes preparation from the obtained MSCs, wherein the MSC preparation is created by altering the growth conditions to create a specific product to match the specific pathology of the recipient by thorough characterization and proteomic analysis of the growth factors and molecular characterization of the exosome RNA present in the growth media to maximize the treatment efficacy by matching the product to the exact pathology identified and needing to be treated. The autogenic or allogenic MSC derived exosomes may then be administered to a patient via IV infusion.

While the composition is described above as including MSCs, the use of other fibroblast-like cells is envisioned. The product may contain keratinocytes or melanocytes. The MSCs may be derived from multiple sources such as bone marrow stroma, adipose, blood, dermis, periosteum, bone, and other tissues. In embodiments, the MSCs may be derived from the patient to which the composition will be applied (autologous) or derived from another individual (allogeneic). The MSCs may be culture expanded to collect the conditioned media or to increase the quantity of cells for the lysate or used freshly prior to incorporation into the composition of the present disclosure.

As mentioned above, the acellular MSC derived growth factors and exosomes may be dissolved, mixed, or suspended into a mixture suitable for IV infusion to create an infusion product, wherein a therapeutic dosage of such infusion product may be administered via an IV to a patient. The infusion product may be used for the treatment of various medical conditions, including a cancer or metastasis, aplastic anemia, Plasma cell disorders, Inborn errors of metabolism, immune deficiencies, POEMS syndrome, Fibromyalgia, Multiple sclerosis, Parkinson's disease, Crohn's disease, Spinal cord injury, Acute orthopedic soft tissue injuries, Neuropathy, Liver pathology due to alcohol use, Lyme disease, primary amyloidosis, and the like.

### C. Mesenchymal Stem Cells

As noted throughout, the treatment compositions disclosed herein can utilize exosomes and/or growth factors derived from mesenchymal stem cells (MSCs). While existing autogenous and allogeneic MSCs contained within bone marrow, bone marrow concentrate, synovia-derived mesenchymal stem cells (MSCs), or adipose-derived stromal vascular fraction (SVF) or various post-natal products from umbilical cord, placenta or amnion, expanded MSC cultures are currently being used to treat wounds, orthopedic pathology, and spine pathology; the existing treatments do not contain large amounts of MSC secretomes (including, but not limited to growth factors, cytokines, chemokines, exosomes, extracellular vesicles, and/or extracts). Additionally, despite evidence in the art that treatments comprising stem cells (including injectable treatments) can help prevent aging and treat scarring, uneven pigmentation, existing skin products, such as creams, lotions, serums, make-up, and the like, while including ingredients that potentially help treat and strengthen the skin, other topical products do not penetrate the epidermis and more importantly do not include human MSCs, or MSC-derived growth factors and proteins. In fact, prior to the present disclosure an active MSC growth factor product that can be used for these applications has not been developed. Thus, in one aspect, disclosed herein are therapeutic IV compositions (including, but not limited to MSC derived growth factor, MSC derived exosomes, MSC extracts and/or extracellular vesicle comprising compositions) for use in the treatment of a cancer or metastasis, aplastic anemia, Plasma cell disorders, Inborn errors of metabolism, immune deficiencies, POEMS syndrome, Fibromyalgia, Multiple sclerosis, Parkinson's disease, Crohn's disease, Spinal cord injury, Acute orthopedic soft tissue injuries, Neuropathy, Liver pathology due to alcohol use, Lyme disease, or primary amyloidosis, said treatment compositions comprising (i) a therapeutic IV composition and (ii) a pharmaceutically acceptable carrier.

As noted above, MSC are multipotent cells that have the ability to differentiate into a multitude of cell types including myocytes, chondrocytes, adipocytes, and osteoblasts. Typically, these cells can be found in the placenta, umbilical cord blood, adipose tissue, bone marrow, or amniotic fluid, including perivascular tissue. As used herein, “MSC” refers to non-terminally differentiated cells including but not limited to multipotential stem cell, multipotential stromal cell, stromal vascular cells, pericytes, perivascular cells, stromal cells, pluripotent cells, multipotent cells, adipose-derived fibroblast-like cells, adipose-derived stromal vascular fraction, adipose-derived MSC, bone marrow-derived fibroblast-like cells, bone marrow-derived stromal vascular fraction, bone marrow-derived MSC, tissue-derived fibroblast-like cells, adult stem cells, adult stromal cells, keratinocytes, and/or melanocytes.

It has been long recognized that MSC, in addition to their differentiation potential, have the immunomodulatory abilities resulting in the expression of many different cytokines and growth factors. As used herein, a “therapeutic IV composition” refers to a composition comprising MSC derived growth factors, MSC derived exosomes, extracellular vesicles, or acellular extracts of MSCs or MSC lysates obtained from human MSCs, fibroblast-like cells, and non-human animal MSCs including, but not limited to MSCs from horses, cows, pigs, sheep, non-human primates, dogs, cats, rabbits, rats, and mice. In embodiments, the MSCs may be derived from the patient to which the composition will be applied (autologous) or derived from another individual (allogeneic). The MSCs may be culture expanded to collect the conditioned media or to increase the quantity of cells for the lysate or used freshly prior to incorporation into the composition of the present disclosure.

The therapeutic IV composition (including, but not limited to MSC derived growth factors, MSC derived exosomes, MSC derived extracts and/or extracellular vesicle comprising compositions) may comprise about 0.00001 to about 20 wt. %, such as from about 0.01 to about 10 wt. %, of a mesenchymal stem cell (MSC) extract, MSC derived exosome, and/or MSC derived growth factor. The therapeutic IV composition may comprise either MSC conditioned media or MSC lysate from cell culture expanded MSCs. In some embodiments, the composition may further comprise from about 0.01 to about 10 wt. % of a cell-free medium conditioned by growth of MSCs or MSC lineage cells, wherein the cells are cultured under normal hyperoxyic culturing conditions or under artificial wound healing conditions.

As used herein, MSC growth factors include but are not limited to prostaglandin E2 (PGE2), transforming growth factor β1 (TGF-β1), hepatocyte growth factor (HGF), stromal cell derived factor-1 (SDF-1), nitric oxide, indoleamine 2,3-dioxygenase, interleukin-4 (IL-4), IL-6, interleukin-10 (IL-10), IL-1 receptor antagonist and soluble TNF-α receptor, insulin-like growth factors, fibroblast growth factors (FGF) 1-23 (especially, FGF1 and FGF2), bone morphogenetic proteins (BMPs) 1-15, epidermal growth factor (EGF), transforming growth factor-α (TGF-α) macrophage-stimulating protein (MSP), platelet derived growth factor (PLGF), vascular endothelial growth factor (VEGF), macrophage colony stimulating factor (M-CSF), insulin, granulocyte colony stimulating factor (G-CSF), granulocyte macrophage colony stimulating factor (GM-CSF), as well as hormones including estrogen, and thyroid hormones.

In one aspect, the therapeutic IV composition comprises MSC growth factors, MSC exosomes, and/or cellular extracts of MSCs or MSC lysates obtained from MSCs cultured under standard hyperoxyic culturing conditions (for example, 21% oxygen) or MSCs cultured under artificial wound healing conditions (such as, for example, 0.1% to about 5% oxygen in the presence of inflammatory cytokines, angiogenic factors, and reduced glucose).

As disclosed herein artificial wound healing conditions simulate growth conditions in real wounds where there is a reduction in nutrient supply and reduction of waste removal that is usually caused by a disruption in local blood circulation. This creates a harsh environment for cells until new blood vessels are created and blood circulation is restored. Accordingly, artificial wound healing conditions used to culture MSCs can include one or more of the following growth conditions reduction in glucose availability, reduction in oxygen tension, reduction in pH, and increased temperature.

In one aspect, the glucose availability can be reduced relative to normal control. Modified culture media to reduce glucose, but not damage the cells can be between 0 and 50% reduction in glucose, more preferably between about 5% and 40% reduction in glucose. For example, MSC artificial wound healing culture conditions can comprise glucose reduction of about 1, 2, 3, 4, 5, 6,7 ,8 9, 10, 11, 12,13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% such as a glucose reduction from about 5% to about 15%, from about 10% to about 20%, from about 15% to about 25%, from about 20% to about 30%, or from about 25% to about 35%.

In one aspect, oxygen tension can be reduced to oxygen levels to hypoxic conditions. Normal atmospheric oxygen is approximately 21% and any reduction is considered hypoxic. Thus, in one aspect, MSCs can be cultured at between 0.0% and 20.9% oxygen, from about 0.1% to about 0.5% oxygen, from about 0.1% to about 2.0%, from about 0.1% to about 5.0% oxygen, from about 0.5% to 5.0%, from about 1.0% to about 10% oxygen, about 5.0% to about 10.0% oxygen; and from about 10.0% to about 15.0% under artificial wound healing conditions. Preferably during MSC would healing culture conditions oxygen tension is between about 0.5% and 20.5% oxygen, such as, for example, 0, 0.05, 0.1, 0.2, 0.3, 0.4, 0.5, 0.6, 0.7, 0.8, 0.9, 1.0, 1.1, 1.2, 1.3,1.4, 1.5, 1.6, 1.7, 1.7, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, or 20.5% oxygen.

The pH can also be reduced under artificial wound healing conditions. Physiologic pH is maintained very tightly and is usually very close to a neutral pH=7.2±0.2 (7.0-7.4). However, in a wound the acidic environment can have a pH=6.2±0.2 (i.e., a pH from 6.0 to about 6.4). Thus, under artificial wound healing culture conditions, pH can be from about 6.0 to about 7.4, for example, from 6.0 to about 6.4, from about 6.2 to about 6.4, from about 6.2 to about 6.6, from about 6.4 to about 6.6, from about 6.4 to about 6.8, or from about 6.6 to about 7.0, such as 6.0, 6.1, 6.2, 6.3, 6.4, 6.5, 6.6, 6.7, 6.8, 6.9, 7.0, 7.1, 7.2, 7.3 or 7.4.

Under artificial wound healing culture conditions, the temperature of the culture environment may be raised to simulate temperature increases at the site of a wound. Physiologic homeostasis temperature is maintained at 37° C. (98.6° F.). A slight increase or decrease can cause significant changes to cellular metabolism. By increasing the temperature above 37° C. to any temperature up to about 40° C. (104° F.) can create an “feverous” environment. Thus, in on aspect, the artificial wound healing culture conditions for the MSCs can comprise from about 35° C. to about 39° C., from about 35° C. to about 36° C., from about 36° C. to about 37° C., from about 37° C. to about 38° C., from about 38° C. to about 39° C., from about 39° C. to about 40° C. In one aspect, the temperature of the artificial wound healing culture can be 35.0, 35.1, 35.2, 35.3, 36.4, 35.5, 35.6, 35.7, 35.8, 35.9, 36.0, 36.1, 36.2, 36.3, 36.4, 36.5, 36.6, 36.7, 36.8, 36.9, 37.0,.37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, or 40.0° C.

In one aspect, it is understood and herein contemplated that one way to treat a wound is through administration of the MSC secretome compositions (including, but not limited to MSC growth factor, MSC exosome, MSC extracts and/or extracellular vesicle comprising compositions) subcutaneously, intramuscularly, intravenously, topically (such as, for example, through the use of salves, creams, and/or ointments), but also by impregnating stents, sponges, matrixes, scaffolds, bandages, dressing, sutures, grafts, surgical drapes, surgical adhesive, and/or staples with the MSC secretome compositions. Thus, in one aspect, disclosed herein are medicated stents, scaffolds, sponges, matrixes, adhesive bandages, wound dressings, grafts, surgical drapes, sutures, salves, creams, or wound adhesives comprising a therapeutically effective amount of the MSC secretome composition. The MSC secretome compositions (including, but not limited to MSC growth factor, MSC exosome, MSC extracts and/or extracellular vesicle comprising compositions), as noted above, can be administered topically and applied to the face, the neck, the hands, or any other desired part of the body. When applied to an adhesive bandage, wound dressing, grafts, surgical drape, suture, scaffold, matrix, sponge, or stent, the MSC secretome composition can be a applied as a powder.

In one aspect, the MSC secretome compositions (including, but not limited to MSC growth factor, MSC exosome, MSC extracts and/or extracellular vesicle comprising compositions)disclosed herein may comprise any known ingredients typically found in the wound healing fields, such as oils, waxes or other standard fatty substances, or conventional gelling agents and/or thickeners; emulsifiers; moisturizing agents; emollients; sunscreens; hydrophilic or lipophilic active agents, such as ceramides; agents for combating free radicals; bactericides; sequestering agents; preservatives; basifying or acidifying agents; fragrances; surfactants; fillers; natural products or extracts of natural product, such as aloe or green tea extract; vitamins; or coloring materials. Other ingredients that may be combined with the powder may include an antioxidant, which can be selected from a variety of antioxidants. Suitable antioxidants include vitamins, such as Vitamin C (L-Ascorbate, Ascorbate-2 Phosphate magnesium salt, Ascorbyl Palmitate, Tetrahexyldecyl Ascorbate), Vitamin E (Tocotrienol), Vitamin A (retinol, retinal, retinoic acid, provitamin A carotenoids, such as beta-carotene), N-acetyl glucosamine, or other derivatives of glucosamine. Other ingredients may include at least one essential fatty acid, such as Ω-3, Ω-6, and Ω-9 polyunsaturated fatty acids, such as linoleic acid (LA), gamma-linoleic acid (GLA), alpha-linoleic acid (ALA), dihomo-y-linolenic acid (DGLA), arachidonic acid (ARA), and others. The fatty acids may be derived from various sources including evening primrose oil, black currant oil, borage oil, or GLA modified safflower seeds. Other ingredients may include a platelet rich fibrin matrix, at least one ingredient to support ECM production and production of hyaluronic acid, such as N-acetyl glucosamine or other derivatives of glucosamine, ultra-low molecular weight (ULMW) hyaluronic acid, chondroitin sulfate, or keratin sulfate.

### D. Methods of Treating Disease

A bone marrow transplant is a procedure that infuses healthy blood-forming stem cells into your body to replace your damaged or diseased bone marrow. A bone marrow transplant is also called a stem cell transplant. A patient might need a bone marrow transplant if their bone marrow stops working properly and does not produce enough healthy blood cells. Bone marrow transplants currently either use cells from your own body (autologous transplant) or from a donor (allogeneic transplant). These bone marrow transplants can benefit people with a variety of both cancerous (malignant) and noncancerous (benign) diseases, including, but not limited to Acute leukemia, Adrenoleukodystrophy, Aplastic anemia, Bone marrow failure syndromes, Chronic leukemia, Hemoglobinopathies, Hodgkin's lymphoma, Multiple myeloma, Myelodysplastic syndromes, Neuroblastoma, Non-Hodgkin's lymphoma, Plasma cell disorders, POEMS syndrome, and/or Primary amyloidosis.

The ability to provide cell-free bone marrow transplantation (signals) and treat these diseases through IV infusion can restore the patient's immune function as well at treat these diseases. Exosomes can home to site of inflammation and/or injury and work to reprogram local cells to be active in restoration and regeneration of diseased or damaged tissues, including spinal cord damage or even neural degenerative diseases. The delivery of stem cells to treat these issues is limited because the cells cannot pass the blood-brain barrier and will most likely be trapped within the capillaries of the lungs.

The systemic delivery of stem cell signals (growth factors, cytokines and exosomes derived from MSC) can home to sites of injury and provide their regenerative signals directly to the damaged or diseased cells. This can work to treat Fibromyalgia, Multiple sclerosis, Parkinson's disease, Crohn's disease, Spinal cord injury, Acute orthopedic soft tissue injuries, Neuropathy, Liver pathology due to alcohol use, Lyme disease. Accordingly, in one aspect, disclosed herein are methods of treating, inhibiting, reducing, ameliorating and/or preventing a disease, disorder, injury (such as, for example, a cancer or metastasis, aplastic anemia, Plasma cell disorders, Inborn errors of metabolism, immune deficiencies, POEMS syndrome, Fibromyalgia, Multiple sclerosis, Parkinson's disease, Crohn's disease, Spinal cord injury, Acute orthopedic soft tissue injuries, Neuropathy, Liver pathology due to alcohol use, Lyme disease, or primary amyloidosis) in a subject comprising administering to a subject a therapeutically effective amount of a mesenchymal stem cell (MSC) therapeutic IV composition.

In one aspect, disclosed herein are methods of treating, inhibiting, reducing, ameliorating and/or preventing a disease, disorder, injury (such as, a cancer or metastasis, aplastic anemia, Plasma cell disorders, Inborn errors of metabolism, immune deficiencies, POEMS syndrome, Fibromyalgia, Multiple sclerosis, Parkinson's disease, Crohn's disease, Spinal cord injury, Acute orthopedic soft tissue injuries, Neuropathy, Liver pathology due to alcohol use, Lyme disease, or primary amyloidosis) in a subject, wherein the therapeutic IV composition comprises MSC derived exosomes and/or growth factors (such as, for example, prostaglandin E2 (PGE2), transforming growth factor β1 (TGF-β1), hepatocyte growth factor (HGF), stromal cell derived factor-1 (SDF-1), nitric oxide, indoleamine 2,3-dioxygenase, interleukin-4 (IL-4), IL-6, interleukin-10 (IL-10), IL-1 receptor antagonist and soluble TNF-α receptor, insulin-like growth factors, fibroblast growth factors (FGF) 1-23 (especially, FGF1 and FGF2), bone morphogenetic proteins (BMPs) 1-15, epidermal growth factor (EGF), transforming growth factor-α (TGF-α) macrophage-stimulating protein (MSP), platelet derived growth factor (PLGF), vascular endothelial growth factor (VEGF), macrophage colony stimulating factor (M-CSF), insulin, granulocyte colony stimulating factor (G-CSF), granulocyte macrophage colony stimulating factor (GM-CSF), and/or hormones including estrogen, and thyroid hormones) obtained from MSC.

It is understood and herein contemplated that the disclosed MSC exosome treatments may not be curative of a disease, disorder, injury, or condition, but may reduce or inhibit the injury, disease, or disorder. The physical pain associated with these diseases are caused primarily from local inflammation. These MSC signals are known to be anti-inflammatory which can cause a reduction in local pain. Being able to deliver these signals via intravenous (IV) delivery will allow the signals to home to these local sites of inflammation and act directly to treat the local cells.

In several experimental injury models, including stroke, myocardial infarction, liver toxicity, kidney disease, and status epilepticus, the therapeutic effects of systemic MSC delivery could be replicated by transplantation of exosomes produced and secreted by MSCs. Furthermore, MSC exosomes have been shown to modulate immune function in vivo as well as to promote cortical neurite outgrowth and endothelial cell proliferation, migration, and tube formation in vitro, indicating that MSC exosomes might be capable of mediating many of the histological changes observed after intravenous MSC infusion in spinal cord injury (SCI) animals. We therefore postulated that exosomes might be the secreted factors responsible for the therapeutic effects of IV infused MSCs on SCI recovery.

In one aspect, the therapeutic IV composition decreases symptoms of a disease, disorder, and/or injury (such as, for example, pain, inflammation, and/or swelling) rather than being curative or repairing the disease, disorder, or injury. Thus, in one aspect, disclosed herein are methods of treating, inhibiting, reducing, preventing and/or ameliorating any symptom including, but not limited to pain, inflammation, and/or swelling associated with a disease, disorder, and/or injury (such as, for example, a cancer or metastasis, aplastic anemia, Plasma cell disorders, Inborn errors of metabolism, immune deficiencies, POEMS syndrome, Fibromyalgia, Multiple sclerosis, Parkinson's disease, Crohn's disease, Spinal cord injury, Acute orthopedic soft tissue injuries, Neuropathy, Liver pathology due to alcohol use, Lyme disease, or primary amyloidosis) comprising administering to the subject any of the therapeutic IV composition disclosed herein (said compositions comprising MSC derived exosomes and/or MSC derived growth factors).

### E. REFERENCES

AskMayoExpert. Hematopoietic stem cell transplant. Rochester, Minn.: Mayo Foundation for Medical Education and Research; 2015.
Blood and marrow stem cell transplantation. Leukemia & Lymphoma Society. http://www.lls.org/resource-center/download-or-order-free-publications. Accessed Jul. 8, 2016.
Lankford K L, Arroyo E J, Nazimek K, Bryniarski K, Askenase P W, Kocsis J D (2018) Intravenously delivered mesenchymal stem cell-derived exosomes target M2-type macrophages in the injured spinal cord. PLoS ONE 13(1): e0190358.

1. A therapeutic intravenous (IV) infusion composition for the treatment of various medical conditions, the therapeutic IV infusion comprising: acellular mesenchymal stem cell (MSC) derived growth factors and/or exosomes.

2. The therapeutic IV infusion of claim 1, wherein the acellular MSC derived growth factors and exosomes are obtained from cells or cell conditioned media cultured under normal hyperoxic culturing conditions and cells cultured under harsh wound healing conditions.

3. A method of treating, reducing, inhibiting, ameliorating, and/or preventing a cancer or metastasis, aplastic anemia, Plasma cell disorders, Inborn errors of metabolism, immune deficiencies, POEMS syndrome, Fibromyalgia, Multiple sclerosis, Parkinson's disease, Crohn's disease, Spinal cord injury, Acute orthopedic soft tissue injuries, Neuropathy, Liver pathology due to alcohol use, Lyme disease, or primary amyloidosis in a subject comprising administering to the subject IV infusion composition of claim 1.

4. The method of treating, reducing, inhibiting, ameliorating, and/or preventing a cancer and/or metastasis of claim 3, wherein the cancer comprises acute leukemia, Adrenoleukodystrophy, Aplastic anemia, Bone marrow failure syndromes, Chronic leukemia, Hemoglobinopathies, Hodgkin's lymphoma, Multiple myeloma, Myelodysplastic syndromes, Neuroblastoma, Non-Hodgkin's lymphoma.
